(19) United States
(12) Patent Application Publication
(10) Pub. No.: US 2013/0046333 A1
(43) Pub. Date: Feb. 21, 2013

(54) INTRALUMENAL RETRIEVAL SYSTEM

(71) Applicants: Donald K. Jones, Dripping Springs, TX (US); Vladimir Mitelberg, Austin, TX (US)

(72) Inventors: Donald K. Jones, Dripping Springs, TX (US); Vladimir Mitelberg, Austin, TX (US)

(21) Appl. No.: 13/589,134

(22) Filed: Aug. 18, 2012

Related U.S. Application Data
(60) Provisional application No. 61/525,366, filed on Aug. 19, 2011.

(51) Int. Cl. A61B 17/22

(52) U.S. Cl. 606/200

(57) ABSTRACT

Devices, systems and methods are provided for performing intra-lumenal medical procedures in a desired area of the body. Retrieval devices and methods of performing medical procedures to remove foreign objects to re-establish the intravascular flow of blood are provided.

### CROSS-REFERENCE TO RELATED APPLICATIONS

This application claims the benefit of U.S. Prov. Ser. 61/525,366 filed Aug. 19, 2011 which is hereby incorporated by reference herein in its entirety.

### BACKGROUND OF THE INVENTION

The field of intralumenal therapy for the treatment of vascular disease states has for many years focused on the use of many different types of therapeutic devices. While it is currently unforeseeable that one particular device will be suitable to treat all types of vascular disease states it may however be possible to reduce the number of devices used for some disease states while at the same time improve patient outcomes at a reduced cost. To identify potential opportunities to improve the efficiency and efficacy of the devices and procedures it is important for one to understand the state of the art relative to some of the more common disease states.

One cerebrovascular disease state is ischemia resulting from reduced or blocked arterial blood flow. The arterial blockage may be due to thrombus, plaque, foreign objects or a combination thereof. Generally, soft thrombus created elsewhere in the body (for example due to atrial fibrillation) that lodges in the distal cerebrovasculature may be disrupted or dissolved using mechanical devices and or thrombolytic drugs. While guidewires are typically used to disrupt the thrombus, some sophisticated thrombectomy devices have been proposed. For instance U.S. Pat. No. 4,762,130 to Fogarty et al., entitled, “Catheter with Corkscrew-Like Balloon”, U.S. Pat. No. 4,998,919 of Schepp-Pesh et al., entitled, “Thrombectomy Apparatus”, U.S. Pat. No. 5,417,703 to Brown et al., entitled “Thrombectomy Devices and Methods of Using Same”, and U.S. Pat. No. 6,663,650 to Sepetka et al., entitled, “Systems, Methods and Devices for Removing Obstructions from a Blood Vessel” discloses devices such as catheter based corkscrew balloons, baskets or filter wires and helical coiled retrievers. Commercial and prototype versions of these devices have shown only marginal improvements over guidewires due to an inability to adequately grasp the thrombus or to gain vascular access distal to the thrombus(i.e. distal advancement of the device pushes the thrombus distally).

To remove foreign objects from a body lumen, a number of catheter based retrieval devices have been proposed such as a spring jaw medical instrument disclosed in U.S. Pat. No. 5,782,747 entitled, “Spring Based Multi-purpose Medical Instrument.” That apparatus, used for capturing an intravascular object, has a variety of jaw structures including a configuration having opposing serrated surfaces for crushing an object and a configuration having a closed loop jaw suitable for grasping an object. Proposed devices for the removal of foreign objects such as embolic coils and stents are disclosed in U.S. Pat. No. 6,989,020 to Jones et al., entitled, “Embolic Coil Retrieval System” which includes biased jaw members having major and minor teeth positioned along the outer edge of the jaw members. Additional retrieval devices are described in U.S. Pat. No. 6,673,100 to Diaz et al., entitled, “Method and Device for Retrieving Embolic Coils” wherein the system includes an outwardly biased jaw member having an acutely angled latch member for grasping coils.

### SUMMARY OF THE INVENTION

In accordance with one aspect of the present invention there is provided a medical device system for removing an object from a body lumen of a mammal. The retrieval system includes a catheter or sheath having proximal and distal ends and a lumen extending therethrough and a retrieval device comprising an elongate flexible member having a capture member coupled to its distal end. The retrieval device is slidably positioned within the catheter lumen. The capture member of the retrieval device has first and second arms that are resiliently biased in an open configuration. The capture member further includes an engagement member extending between the first and second arms. The engagement member includes a plurality of retaining elements positioned along the length of the engagement member which are resiliently biased and generally extend in a direction towards the first or second arms when unconstrained. The capture member of the retrieval device is operable between an unconstrained configuration in which the arms are open and a constrained configuration where the arms are generally closed or collapsed. In the constrained configuration the arms are brought into contact with the retaining elements of the engagement member causing the retaining elements to collapse. During delivery to a target site, the retrieval device is positioned within the lumen of the catheter thereby placing the capture member in a constrained configuration. Upon exiting the catheter lumen, the resilient arms of the capture member are unconstrained and move to an open configuration exposing the engagement member and allowing the retaining elements to take there biased configuration. To capture an object the capture member is positioned such that a portion of the object is between a capture arm and retaining element. The catheter may then be advanced to collapse the capture arms thus securing the object between the arm and engagement member. The retrieval system may then be removed along with the object.

In accordance with another aspect of the present invention there is provided a retrieval system comprising biocompatible resilient materials. Suitable resilient materials include metal alloys such as nitinol, titanium, stainless steel and cobalt chromium and any alloys thereof Additional suitable materials include polymers such as polyimides, polyamides, fluoropolymers, polyetheretherketone (PEEK) and shape memory polymers. These materials may be formed into desired shapes by a variety of methods which are appropriate to the materials be in utilized such as laser cutting, injection molding, welding, electrochemical machining, machining, photo-etching and casting.

In accordance with still another aspect of the present invention there is provided a retrieval system having a capture member that includes radiopaque materials to provide visibility under fluoroscopy. The radiopaque materials may take the form of markers (including coils, rivets and radiopaque shrink tubing) positioned on portions of the capture member. The capture member may include a radiopaque coating utilizing radiopaque materials. Suitable radiopaque materials include gold, tantalum, tungsten, platinum, iodinated or barium containing compounds or mixtures and alloys thereof. Coatings may be applied using known techniques such as electro deposition, sputtering, dipping, printing and spray methods.

In accordance with another aspect of the present invention there is provided a method of retrieving an object, such as an embolic coil or stent from the body. The method comprises the steps of positioning a catheter at a preselected site within a body lumen, providing an elongate flexible retrieval device deliverable through the lumen of said catheter, advancing the retrieval device such that the distal capture member exist the catheter lumen and expands, positioning the capture member to engage the object, advancing the catheter relative to the retrieval device to secure the object and removing the retrieval system and object from the body.

A more detailed explanation of the invention is provided in the following description and claims, and is illustrated in the accompanying drawings.

### BRIEF DESCRIPTION OF THE DRAWINGS

FIG. 1 is a partial cross-sectional view of a retrieval system according to an embodiment of the present invention.

FIG. 2 is an enlarged partial cross-sectional view of the distal end of the retrieval system according to an embodiment of the present invention.

FIG. 3A is a partial perspective view of the distal portion of a deployed retrieval system according to an embodiment of the present invention.

FIG. 3B is a partial perspective view of the distal portion of a deployed retrieval system according to another embodiment of the present invention.

FIGS. 4 through 7 are partial cross-sectional views illustrating a method of using a retrieval system within a vessel at a target site to remove an object according to an embodiment of the present invention.

### DETAILED DESCRIPTION OF THE INVENTION

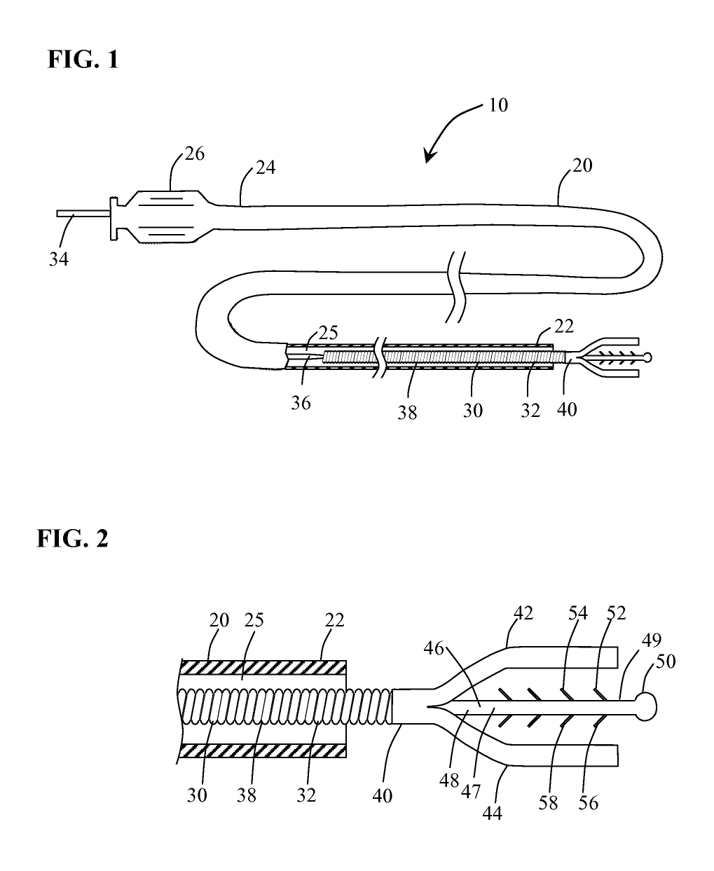

FIG. 1 illustrates a retrieval system 10 suitable for use in removing foreign objects such as embolic coils and stents. Retrieval system 10 includes a catheter 20 formed of a polymeric material as is known in the art, having distal end 22, proximal end 24 and lumen 25 extending therethrough. Catheter hub 26, having a luer connector is coupled to proximal end 24. While not shown, the construction of catheter 20 may utilize known catheter technologies that incorporate braiding and or coiling using metallic or non-metallic reinforcing filamentous materials to provide high strength while maintaining catheter flexibility. The incorporation of lubricious hydrophilic and or hydrophobic materials on the inner and or outer surface of the catheter is considered to be within the scope of known catheter construction techniques and suitable for use in a retrieval system according to embodiments of the present invention. Positioned within catheter 20 is an elongate flexible retrieval device 30 having distal end 32 and proximal end 34. Retrieval device 30 includes a pushable flexible member 36 extending from proximal end 34 to distal end 32. Flexible member 36 preferably takes the form of a wire having a distal taper similar to that of guidewires. Suitable materials include stainless steels, nitinol and polymers. Preferably a flexible coil 38 is positioned over the distal portion of flexible member 36 to aid in delivery. Capture member 40 is coupled to coil 38 at distal end 32 typically through soldering, welding or gluing.

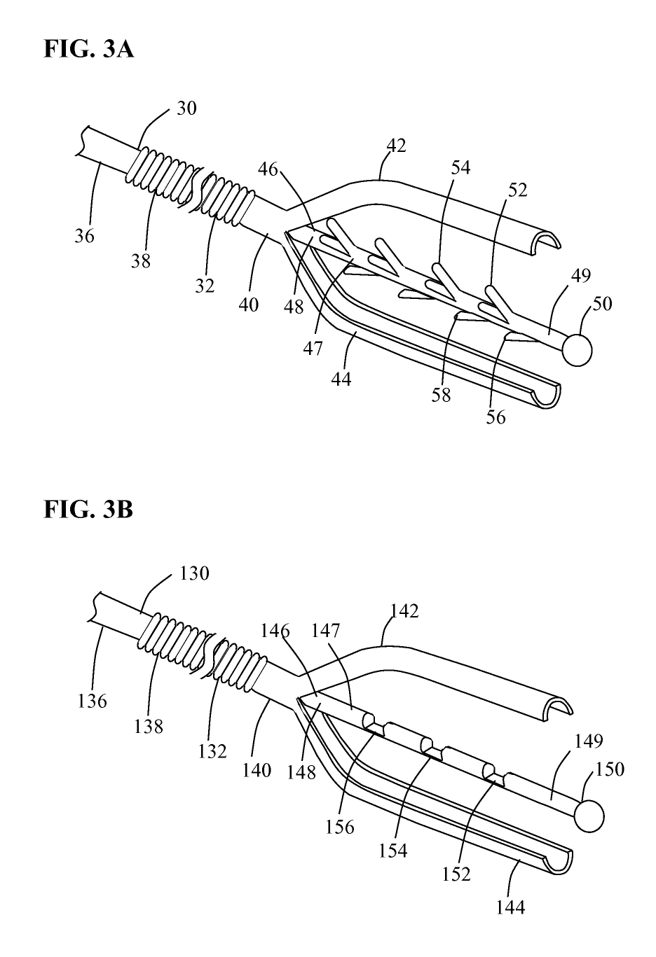

FIG. 2 provides an enlarged view of the distal end of retrieval system 10 where capture member 40 is shown to include capture arms 42 and 44 that extend outwardly from the longitudinal axis of retrieval device 30. Capture arms 42 and 44 are formed of a resilient material and biased in an open configuration. Capture member 40 may be formed from a tube of nitinol that has been partially split wherein each half of the split tube is shaped to become a capture arm. Engagement member 46 having shaft 47, proximal end 48, distal end 49 and distal tip 50 is coupled to capture member 40 at proximal end 48 and is positioned between capture arms 42 and 44. Engagement member 46 includes a number of retaining elements represented by retaining elements 52, 54, 56 and 58. Retaining elements of engagement member 46 extend from shaft 47 in an angled direction towards the capture arms. For instance, representative retaining elements 52 and 54 on one side of shaft 47 are angled towards capture arm 42 while representative retaining elements 56 and 58 on the opposite side of shaft 47 are angled towards capture arm 44. The orientation of the retaining elements relative to the capture arms is depicted in the perspective view of FIG. 3A.

As shown in FIG. 3A, arms 42 and 44 are generally spaced apart when capture member 40 is in an unconstrained configuration. The distance between arms 42 and 44 may range from 1.5 mm to 5 mm for devices designed for use in neurovascular applications, however, may range from 1.5 mm to 50 mm for devices designed for use in other body lumens. The lengths of arms 42 and 44 are also dependant upon a particular design for a particular application but typically range from 3 mm to 50 mm. Representative retaining elements 52 and 54 are spaced apart on the same side of engagement member 46 as previously discussed. The length, angle and spacing distance for retaining elements is dependant upon the designed device and dimensions of the object to be removed but may typically range from length of 1 mm to 20 mm, an angle of 5 to 85 degrees and a spacing distance of 0.010 inches to 0.080 inches. Capture member 40 of retrieval device 30 is operable between an unconstrained configuration in which arms 42 and 44 are open and a constrained configuration, such as within a catheter lumen, where the arms are generally closed or collapsed. In the constrained configuration, arms 42 and 44 are brought into contact with retaining elements 52, 54 56 and 58 of engagement member 46 causing the retaining elements to collapse. In the constrained configuration the collapsed retaining elements are shielded by collapsed arms 42 and 44.

FIG. 3B illustrates a retrieval device according to another embodiment. While many portions of retrieval device 130 are similar to previously described retrieval device 30, engagement member 146 does not have retaining elements that project towards the capture arms. Engagement member 146 includes a plurality of retaining elements 152, 154 and 156 that take the form of grooves or recessed areas in shaft 147.

Preferably, the retrieval devices of embodiments of the present invention comprise a biocompatible resilient material. Suitable resilient materials include metal alloys such as nitinol, titanium, stainless steel. Additional suitable materials include polymers such as polyimides, polyamides, fluoropolymers, polyetheretherketone (PEEK) and shape memory polymers. These materials may be formed into desired shapes by a variety of methods which are appropriate to the materials be in utilized such as laser cutting, thermal heat treating, vacuum deposition, electro-deposition, vapor deposition, chemical etching, photo etching, electro etching, stamping, injection molding, casting or any combination thereof. In addition, the biased resiliency of these materials allow a retrieval device with a normally expanded configuration to have a collapsed, small diameter configuration when constrained within a delivery catheter suitable for delivery to a target site and upon being deployed at a target site return to its expanded configuration.

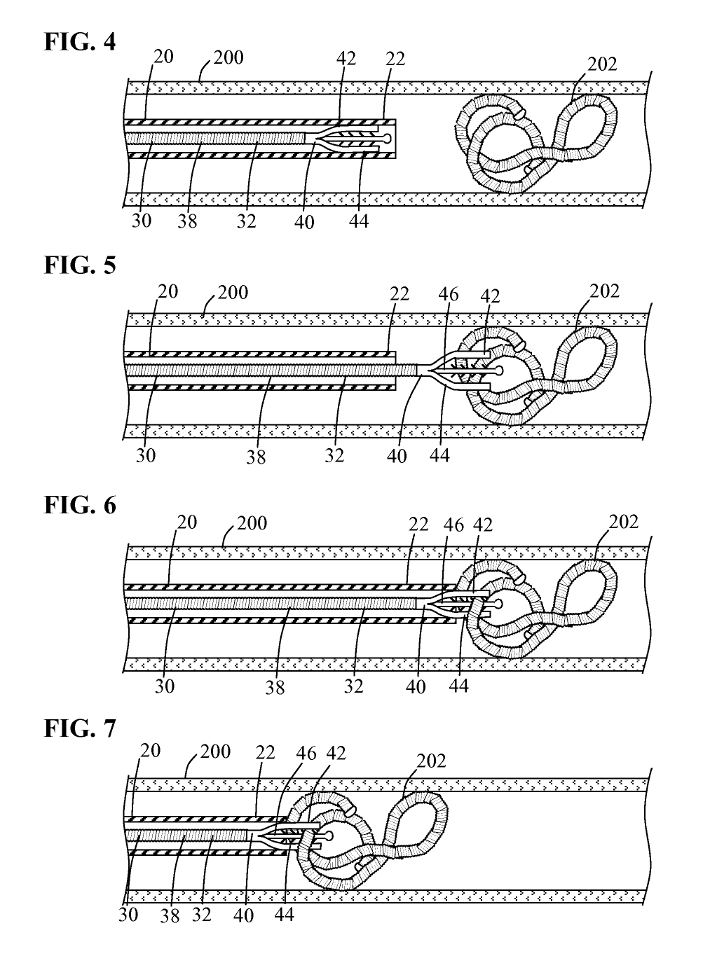

A method of removing a foreign object such as an embolic coil using retrieval system 10 is illustrated in FIGS. 4, 5, 6 and 7. Catheter 20 is introduced into vessel 200 and distal end 22 is positioned adjacent a target site such as embolic coil 202. Retrieval device 30 is then introduced into and advanced through the catheter with the arms 42 and 44 in a constrained collapsed configuration as illustrated in FIG. 4. As shown in FIG. 5, when capture member 40 exits the lumen of catheter 20, arms 42 and 44 move to an open unconstrained configuration. The capture member is then manipulated to cause the retaining elements of engagement member 46 to sufficiently engage a portion of embolic coil 202. Catheter 20 is then advanced relative to retrieval device 30 such that distal end 22 causes arms 42 and 44 to collapse thereby securing a portion of coil 202 as illustrated in FIG. 6. FIG. 7 depicts retrieval system 10, along with secured coil 202, being retracted from the target site and subsequently removed from the body.

Novel devices, systems and methods have been disclosed to remove foreign objects from a body lumen of a mammal. Although preferred embodiments of the invention have been described, it should be understood that various modifications including the substitution of elements or components which perform substantially the same function in the same way to achieve substantially the same result may be made by those skilled in the art without departing from the scope of the claims which follow.

What is claimed is:

1. A medical device for retrieving objects in a patient comprising:
an elongate tubular sheath having distal and proximal ends and a lumen extending therethrough,
an elongate flexible member having distal and proximal ends defining a longitudinal axis and being positioned within the lumen of said sheath,
a capture element coupled to the distal end of said flexible member, said capture element having a pair of resilient capture arms biased outwardly from said longitudinal axis and an engagement member positioned between said capture arms, said engagement member having a plurality of engagement elements spaced apart and extending outwardly from said engagement member.

2. The medical device of claim 1 wherein said capture element comprises nitinol.

3. The medical device of claim 1 wherein said capture element includes a radiopaque material.

4. The medical device of claim 1 wherein said engagement elements extend in a generally proximal direction.

5. The medical device of claim 4 wherein said engagement elements are oriented substantially parallel to the plane defined by said capture arms.

6. A medical device for retrieving objects in a patient comprising:
an elongate tubular sheath having distal and proximal ends and a lumen extending therethrough,
an elongate flexible member having distal and proximal ends defining a longitudinal axis and being positioned within the lumen of said sheath,
a capture element coupled to the distal end of said flexible member, said capture element having a pair of resilient capture arms biased outwardly from said longitudinal axis and an engagement member positioned between said capture arms, said engagement member having a plurality of engagement elements spaced apart along the length of said engagement member.

7. The medical device of claim 6 wherein said capture element comprises nitinol.

8. The medical device of claim 6 wherein said capture element includes a radiopaque material.

9. The medical device of claim 6 wherein said engagement elements extend in a generally proximal direction.

10. The medical device of claim wherein said engagement elements are oriented substantially parallel to the plane defined by said capture arms.
